(19) United States
(12) Patent Application Publication
(10) Pub. No.: US 2008/0164254 A1
(43) Pub. Date: Jul. 10, 2008

(54) Arrangement of Containers Containing Substances for a Dental Ceramic Product

(76) Inventor: Ernst Hegenbarth, Bruchkobel (DE)

Correspondence Address: KNOBBE MARTENS OLSON & BEAR LLP, 2040 MAIN STREET, FOURTEENTH FLOOR, IRVINE, CA, 92614, US

(21) Appl. No.: 11/597,701

(22) PCT Filed: May 26, 2005
(86) PCT No.: PCT/IB2005/001447

§ 371 Date: Sep. 26, 2007

(30) Foreign Application Priority Data
May 26, 2004 (SE) 0401357-9

(51) Int. Cl. A61C 19/10; A61B 19/00; B65D 21/02

(52) U.S. Cl. 220/23.83; 206/63.5; 433/26

(57) ABSTRACT

An arrangement comprises bottle-shaped containers (3) containing various substances for construction and coloring of dental components. The containers are arranged in sector-shaped units which comprise containers with substances belonging to an allocated group of shades. The containers/substances in each respective group are further divided into user categories. Containers/substance related to a first use are placed outermost in the sector-shaped unit, a second user category is placed to the inside of the first user category, and so on. The novel arrangement enhances the efficiency of use of commercially available substances for construction of said products. Several users can use the arrangement simultaneously. The novel arrangement permits natural reproduction of the products in question.

The present invention relates to an arrangement of bottle-shaped containers containing various substances for the construction and coloring of dental products, for example in the form of front teeth. The invention can also apply to other types of products, for example caps and spacer sleeves. The products/teeth can be made of aluminum oxide, titanium oxide, zirconium oxide, etc.

It is already known to arrange substances for construction and coloring of dental ceramic teeth and products of said type in box-shaped units that can be opened and closed and in which, when the box or the like is in the open position, various containers, tubes or tins, instruments, etc., can be exposed and selected from by a user. Reference may be made in this connection to U.S. patent specifications 5,261,815 and 5,588,834.

There is a need to offer an arrangement with better presentation and enhanced user-friendliness of the substances in question and of their different shades. Among other things, there is a need for new esthetically pleasing concepts and variations in the type of work in question here. An aim is to initiate new ideas in connection with use, training, customer service, communication between manufacturer and user, and problems connected with the construction and coloring of the teeth or products. Among other things, the use of the arrangement should be developed so that, for example, individuals with different backgrounds and different levels of knowledge of the construction of the product can use the arrangement at the same time. Despite the above requirements, another aim is to ensure simplified handling in connection with the product constructions according to the above. New substances and components arising in this field must be able to be incorporated into the arrangement. An important requirement of the invention is to permit production of, inter alia, ceramic teeth with a natural appearance through using color harmonies and color shades in connection with production. It is an object of the present invention to solve all or some of the problems indicated above.

That which can principally be regarded as characterizing an arrangement according to the invention is that the bottle-shaped containers are arranged in sector-shaped units which each comprise containers with substances belonging to an allocated group or snades which differs from another group or other groups of shades. The arrangement is additionally characterized in that the containers/substances included in each respective group are divided into user categories, for example categories called Basic Kit, Professional Kit and Stain Kit. The arrangement is additionally characterized in that the containers/substances related to a first use are placed outermost in the sector-shaped unit in question, a second user category is placed to the inside of the first user category in the sector-shaped unit, and so on.

The sector-shaped units can form part of a cone-shaped or pyramid-shaped unit or of a cone-shaped unit with straight side surfaces in a number which differs from the pyramid-shaped unit. Moreover, the outermost containers can be arranged around the unit at a first level. To the inside of the outermost containers, other containers can be arranged around the unit at a second level, and so on. Alternatively, the sector-shaped units can be arranged in a common plane in which every second sector-shaped unit is directed such that the unit as a whole forms a rectangle or square. In an alternative embodiment, the unit can be designed to hang on a wall or similar.

In a further embodiment, each sector-shaped unit can be divided in its longitudinal direction into two or more arc-shaped unit parts which are mutually rotatable about a center axis in the cone-shaped or pyramid-shaped unit so that, in a first position of rotation, the arc-shaped unit parts form the sector shape and, in one or more other positions of rotation, the various arc-shaped unit parts assume laterally offset positions in relation to one another. In the laterally offset positions of the first arc-shaped unit parts, the other arc-shaped unit parts in all the sector-shaped units can be rotated jointly, in which case the containers/substances for the respective user category are arranged to be rotatable in relation to the containers/substances for another user category or other user categories. The cone-shaped or pyramid-shaped unit can thus comprise or be made up of one, two or more ring-shaped elements which are designed to be rotatable relative to one another. Each ring-shaped element consists of arc-shaped sector parts belonging to a user category which differs from the other user category or user categories, meaning that said adjustability for different users can be achieved. In a preferred embodiment, the level differences between the various rows of containers arranged around the cone-shaped or pyramid-shaped unit are designed to provide exposable surfaces on the containers, which surfaces can support identifiable markings for each substance in the containers, so that the shade in question can be easily identified. In a further embodiment, the containers/substances in each sector-shaped unit are arranged in subsectors which extend alongside one another. One or more containers/substances for a first group of shades in one or more of the sector-shaped unit's user categories can be coordinated with containers/substances in an adjacent group of shades and user category. In one embodiment, the cone-shaped or pyramid-shaped unit is provided with a centrally arranged element which supports a chart or samples of shades of substances used for construction and coloring. The element can be removed from the cone-shaped or pyramid-shaped unit and can be made up of disks or plates comprising sector-shaped parts which can be removed from and fixed to the element. Further embodiments of the novel arrangement are evident from the attached dependent claims.

By means of what is proposed above, a new approach is obtained to the existing substances, and to those under constant development, for creating ceramic products with a natural appearance. These can be created on the basis of experience, research, logic and creativity. All standard colors can be produced with the predicted results. Only a small number of groups of colors, for example six, need to be used to give a youthful dental look. The arrangement functions with external and internal concepts, the internal concepts permitting professional modifications for an almost unlimited number of variations.

A presently proposed arrangement having the features characteristic of the invention is described below with reference to the attached drawings, in which

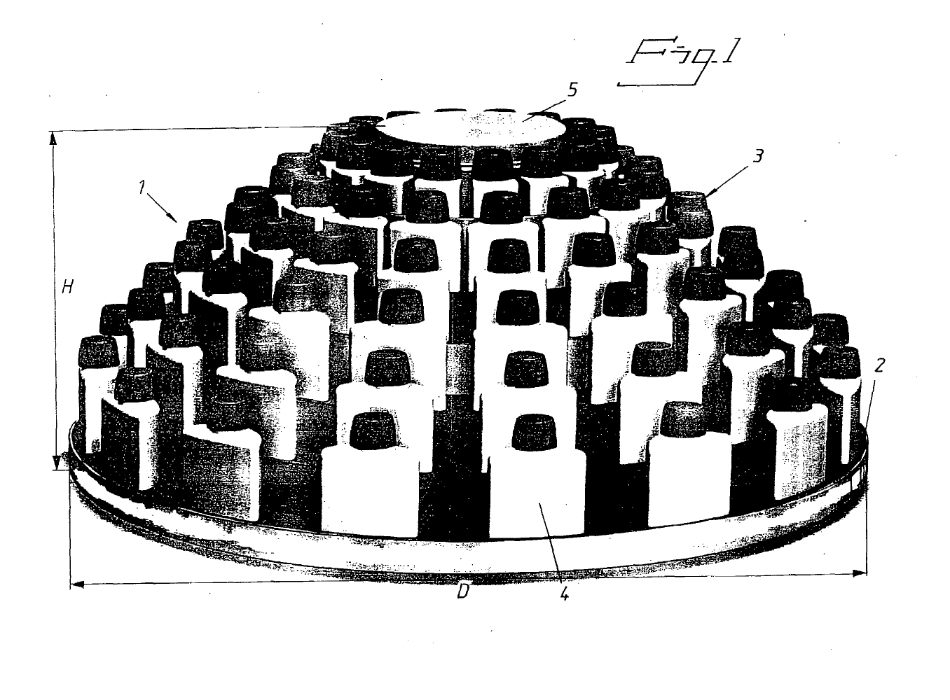

FIG. 1 is a perspective view, obliquely from above, showing an arrangement which comprises a cone-shaped unit on which a number of bottle-shaped units have been arranged in various circular rows and at different levels,

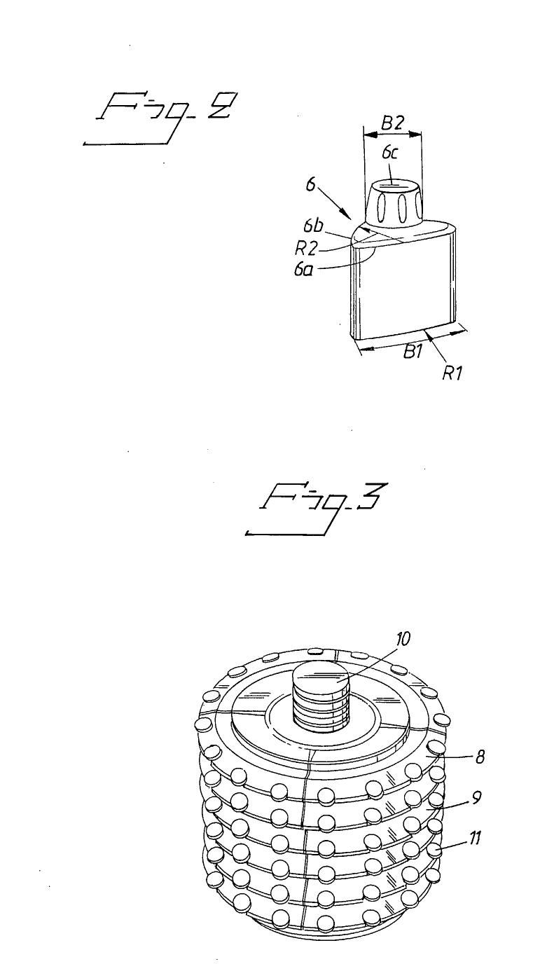

FIG. 2 is a perspective view, obliquely from above, showing an example of the design of a bottle-shaped container according to FIG. 1,

FIG. 3 is a perspective view, obliquely from above, showing inner parts of the element according to FIG. 1, which inner parts support templates or samples in tablet form for different shades used in or on the cone-shaped unit according to FIG. 1,

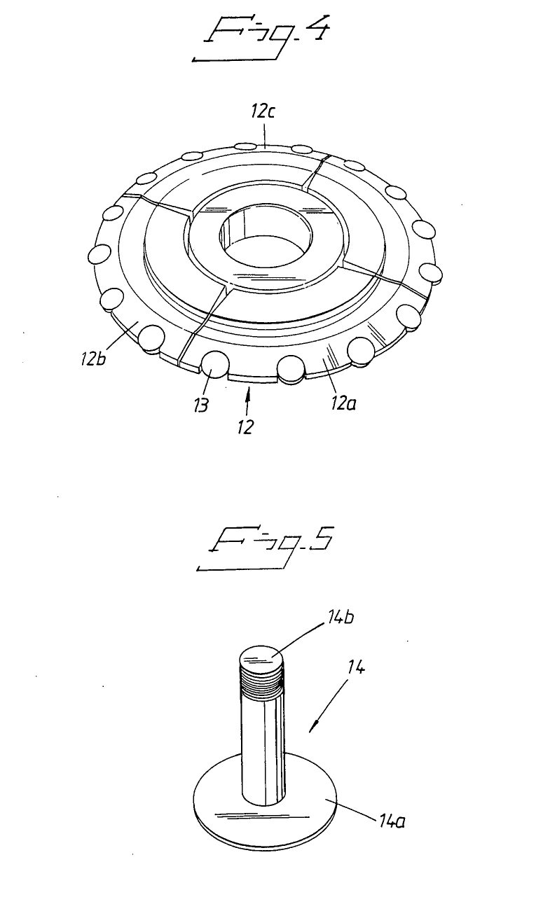

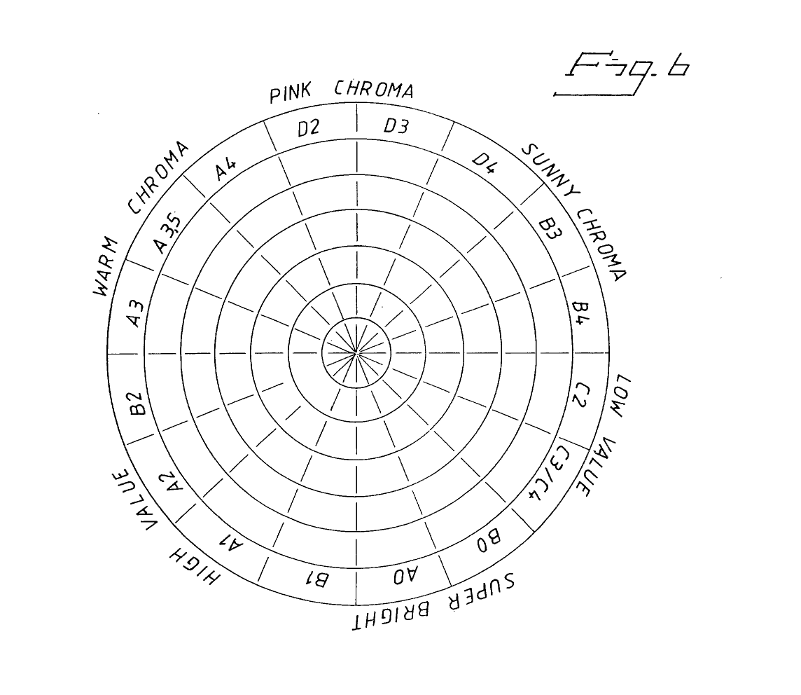

FIG. 4 is a perspective view, obliquely from above, showing a disk-shaped part which is included in the inner parts according to FIG. 3 and which is provided with or comprises sector-shaped disk parts,
FIG. 5 is a perspective view, obliquely from above, showing a bearing element for the element according to FIG. 3,

FIG. 6 is a diagram showing different color shades and user categories, and

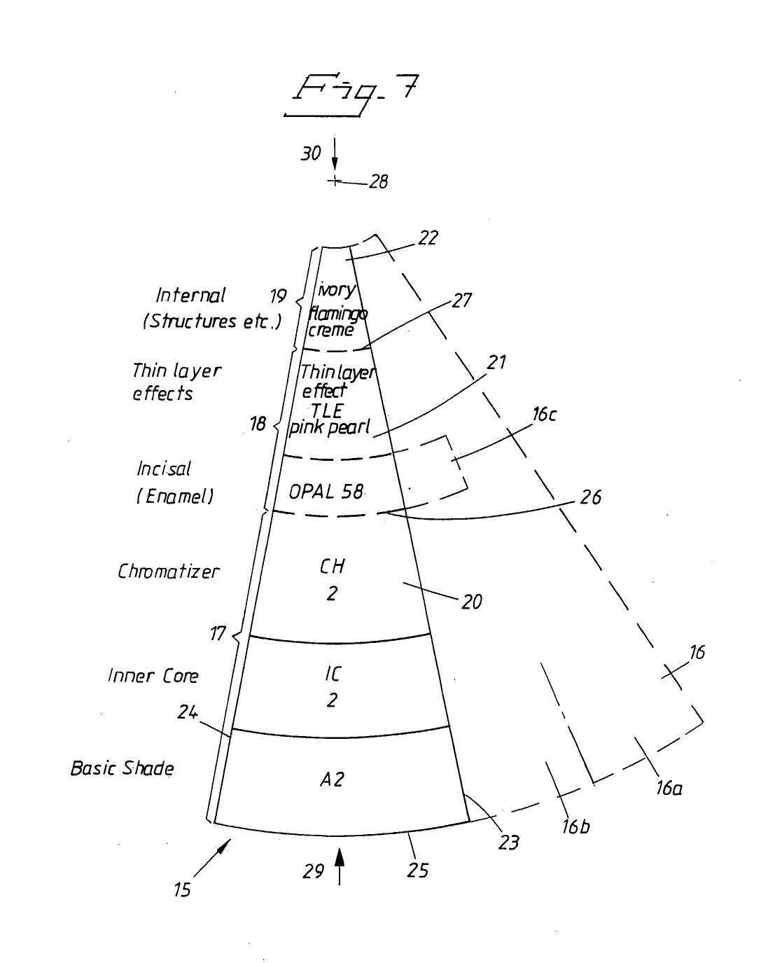

FIG. 7 is a diagram showing a sector-shaped unit included in the arrangement according to FIG. 6.

Reference is also made to the appended table which shows, on the one hand, different user categories and, on the other hand, different shades which are related to the invention.

The cone-shaped unit according to FIG. 1 is indicated generally by 1. The unit has a width or diameter D of 420-500 mm, preferably ca. 460 mm. The unit also has a height H of 140-200 mm, preferably ca. 160 mm. The unit 1 comprises a plate 2 on which a number of containers 3 are disposed in circular rows at different levels. Thus, the containers in the outer circular row are located at a lowest or first level, the containers in the row next to the outermost row are located at a second level, and so on. In this way, sides 4 of the containers are exposed to an observer. The exposed sides can bear identifications for the substances the containers contain. By means of the various levels, the identifications can be easily read off by an observer or user. The various levels can be produced with the aid of shelves arranged on the container 2. Thus, the second row of containers can be set on a circle-shaped shelf with a support surface for the containers which lies higher than the support surface for the containers in the outermost row. The third circular row can be disposed on another circle-shaped shelf whose support surface is arranged at a higher level than the circle-shaped shelf for the second outermost row, and so on. In accordance with the description below, the containers are also arranged on sector-shaped parts. The shelves supporting the containers at different levels can be mutually rotated so that one group of containers in the sector-shaped unit can be observed from one direction and a second group of containers in the sector-shaped unit can be observed from another direction. In this way, the containers can be used at the same time by two or more users.

At its central parts, the cone-shaped unit 1 is provided with an element which is shown in a position lowered into the unit 1. The element can be drawn up out of the unit 1 and, in accordance with what is described below, can have a chart, or samples in tablet shape form, of the shades present in the arrangement. The chart or the tablet-shaped samples can be compared with the markings or identifications on the bottle-shaped containers upon use of the containers and substances.

FIG. 2 shows the design of a bottle-shaped container indicated by 6. The outer side of the container is indicated by 6a and the inner side of the container is indicated by 6b. A radius R1 for the outer side exceeds a radius R2 for the inner side. A closure, for example a cap, is indicated by 6c. The container can be held with the slightly bent fingers and palm of one hand, in which case the inner side 6b fits the “cup shape” of the hand. At the same time, the container can be held in the hand in such a way that the closure 6c can be acted on by the thumb and index finger of the hand. The container according to FIG. 2 has a width B1 in the range of 35-50 mm, preferably the width in two different variants can be ca. 38 mm or ca. 49 mm. The closure 6c has a diameter in the range of 10-20 mm, preferably ca. 15 mm.

The element 5 according to FIG. 1 comprises a casing 7. The element as a whole can be removed from the unit 1 according to FIG. 1. According to FIG. 3, where the casing has been removed, the inner parts of the element consist of a number of disk-shaped or plate-shaped elements, of which two have been labeled in FIG. 3. The disks are mounted on a bearing element 10 and, at their peripheries, the disks or plates support tablet-shaped markings which represent the different shades. In the removed state shown in FIG. 3, the various tabelt-shaped identifications can be compared with the identifications on the container of the unit 1 (see FIG. 1). The disks or plates can be rotated relative to one another.

FIG. 4 shows a plate 12 in the arrangement according to FIG. 3. A comparison is made between its tablet-shaped markings and the conceived dental product with respect to color scale.

FIG. 5 shows a bearing element 14 for said disks or plates. The bearing elements comprise a base part 14a and a cylindrical bearing shaft 14b for the disks/plates.

In accordance with FIG. 6, the containers 3 according to FIG. 1 are arranged in sector-shaped parts, which are six in number in the illustrative embodiment. The sector-shaped parts are called SUPER BRIGHT, HIGH VALUE, WARM CHROMA, PEARL CHROMA, SUNNY CHROMA and LOW VALUE.

In accordance with FIG. 7, the containers are arranged in sector-shaped parts, one sector-shaped part having been indicated by 15 and with solid lines, and another sector-shaped part having been indicated by 16 and broken lines. Each sector-shaped part is divided into user categories, a first user category having been indicated by 17, a second user category by 18, and a third user category by 19. Reference is also made to the appended table which lists a first user category as Basic Kit, Professional Kit and Stain Kit. The user categories can also be divided for straightforward or general use, moderately difficult use, and advanced use. The sector-shaped unit 8 is divided into arc-shaped sector parts 20, 21 and 22, in which each arc-shaped sector part is limited by the side surfaces 23, 24 of the sector unit and circle lines 25, 26 and 27 around the axis of rotation 28 of the sector unit 15, which axis of rotation is common with the center axis of the unit 1 (see FIG. 1). In accordance with the invention, the arc-shaped sector parts 20, 21 and 22 can be mutually rotated. At least the sector part 21 is rotatable in relation to the sector part 20 and the sector part 22 is rotatable in relation to the sector part 21. Containers in, for example, sector part 20 can be viewed from one direction of observation symbolized by 29 in FIG. 8. In the laterally offset positions of the sector parts 21 and/or 22, the containers in each sector part can be viewed from another direction 30 which is different than the direction of observation 29. In this way, different users can use the arrangement at the same time. Each sector, for example sector 16, can be considered as comprising two or more subsector units 16a, 16b which support containers arranged alongside one another in the circular row or rows in question. By means of this arrangement, shades which belong in principle to an adjacent shade group can be related to the shades in the shade group placed alongside. This needs to be the case only in one of the user categories 17, 18 and 19. In the case shown in FIG. 8, containers 16c in sector part 16b are thus regarded as being able to be matched with containers in the same circular row in sector 15, and so on.

The element 5 in the unit 1 can be of telescopic design. Likewise, the unit 1 according to FIG. 1 can be collapsible so that a lower value is obtained for the height H. The targets can have diameters of 6 mm and thicknesses of 2 mm. The disks or plates can be provided with arrangements allowing the tablets to be easily pressed into a recess or removed therefrom. The element 5 can be arranged so as to be rotatable in its bearing according to FIG. 6. Reference is made to the appended table in which the circular rows in the first category comprise Dentines, 6 Liners, 6 Inner Cores, 4 Chromatizers and 8 Opal Enamels, 6 Standard Incisals, Transpa Clear and Occlusal White. Examples of the various shades are shown in the table.

The table also shows that Professional Kit comprises Internal Modifiers, Colored Translucencies, Mother-of-pearl effects, Shoulder Ceramics and Liner Modifiers. The arrangement with Stain Kit comprises Stain Powders, Glaze Powder and Correction Powder.

The invention is not limited to the embodiment shown above by way of example, and instead it can be modified within the scope of the attached patent claims.

TABLE
1. BASIC KIT (48 BOTTLES 20 G)
1st Circle: 16 Dentines

| | |
|---|---|
| Superbright: | B0, A0 |
| High Value: | B1, A1, A2 |
| Warm Chroma: | A3, A3, 5, A4 |
| Pink Chroma: | D2, D3 |
| Sunny Chroma: | B2, B3, B4 |
| Low Value: | C1, C2, C3, 5 |

2nd Circle: 6 Liners, 6 Inner Cores (IC), 4
Chromatizers

| | | |
|---|---|---|
| 6 Liners | 6 Inner Cores | 4 Chromatizers |
| Superbright | IC Superbright | — |
| High Value | IC High Value | — |
| Warm Chroma | IC Warm Chroma | Warm Chroma |
| Pink Chroma | IC Sunny Chroma | Pink Chroma |
| Sunny Chroma | IC Orange Chroma | Sunny Chroma |
| Low value | IC Low Value | Low Value |

3rd Circle: 8 Opal Enamels, 6 Standard Incisals,
Transpa Clear, Occlusal White

Professional Kit (32 bottles, 12 g)
10 Internal Modifiers
Ivory, Flamingo, Creme, Bamboo, Solaris, Safari,
Caramel, Amber, Pearl, Strato
6 Colored Translucencies
4 “Mother-of-pearl” effects
6 Shoulder ceramics
6 Liner Modifiers
Liner High Flu (White)
Liner Pink
Liner Creme
Liner Sunny
Liner Orange
Liner Gray-Violet
Stain Kit (16 Bottles, 3 g)
14 Stain Powders
1 Glaze Powder (glossy pearl)
1 Correction Powder

1. An arrangement comprising a plurality of bottle-shaped containers containing substances for construction and coloring of dental products, and a display unit configured such that the bottle-shaped containers are arranged in sector-shaped units which each comprise containers with substances belonging to an allocated group of shades, which differs from another group of shades, the containers and the respected substance therein included in each respective group are divided into user categories, and in that the containers related to a first use are placed outermost in the respective sector-shaped unit, a second user category is placed to the inside of the first user category in the sector-shaped unit.

2. The arrangement as in claim 1, wherein the display unit is cone-shaped with the outermost containers arranged around the unit at least a first level and a second, higher level, with containers placed to the inside of the outermost containers and arranged around the unit at the second, higher level than the first level.

3. The arrangement as in claim 2, wherein each sector-shaped unit is divided in its longitudinal direction into two or more arc-shaped unit parts which are mutually rotatable about a center axis of the cone-shaped display unit so that, in a first position of rotation, the arc-shaped unit parts form the sector shape and, in one or more other positions of rotation, the various arc-shaped unit parts assume laterally offset positions in relation to one another, and in that, in the laterally displaced positions of first arc-shaped unit parts, the other arc-shaped unit parts in all the sector-shaped units can be rotated jointly, in which case the containers for a respective user category are arranged to be rotatable in relation to the containers for another user category.

4. The arrangement as in claim 2, wherein the cone-shaped unit comprises two or more ring-shaped elements which are rotatable relative to one another, in that each ring-shaped element comprises of arc-shaped sector parts belonging to a user category which differs from the other user category, and the containers for different user categories are rotatably adjustable from different directions of observation seen from the cone-shaped unit.

5. The arrangement as in claim 4, wherein level differences between the various rows of containers arranged around the unit provide exposable surfaces on the containers, which bear identification markings for each substance in the containers.

6. The arrangement as in claim 1, wherein any of patent claims 1-5, characterized in that the containers in each sector-shaped unit are arranged in subsectors which extend alongside one another, and in that one or more containers for a first group of shades and in one or more of the sector-shaped unit's user categories are coordinated with containers in an adjacent group of shades and user category.

7. The arrangement as in claim 2, wherein a first user category is arranged with
a) an outermost circle or first level of containers numbering forty-eight, and allocated to, sixteen dentines,
b) a next outermost row or second level of containers with sixteen liners comprising inner cores, and four chromatizers and
c) a third outermost row or third level comprising eight opal enamels, and six standard incisals, transpa clear and occlusal white.

8. The arrangement as in claim 7, wherein a claimed in any second user category comprises, thirty-two containers in different rows and comprising ten internal modifiers, six colored translucencies, four mother-of-pearl effects, six shoulder ceramics, and six liner modifiers.

9. The arrangement as in claim 8, wherein a third user category comprises, sixteen containers in one or more rows at one or more levels and comprising fourteen stain powders, one glaze powder, and one correction powder.

10. The arrangement as in claim 2, wherein the cone-shaped unit has a width of about 400-450 mm, a height of about 160-220 mm.

11. The arrangement as in claim 2, wherein the cone-shaped unit is provided with a centrally arranged element which supports a chart or samples of shades, used in association with the unit, of substances for construction and coloring.

12. The arrangement as in claim 11, wherein the element (5) can be removed from the cone-shaped unit and supports the chart or samples on its side surfaces.

13. The arrangement as in claim 12, wherein the element is arranged with telescopic parts which can be raised and lowered in relation to the cone-shaped or pyramid-shaped unit.

14. The arrangement as in claim 13, wherein the element comprises disks or plates arranged one on top of the other and on which the chart samples or identifications are arranged, at the peripheries of the disks or plates.

15. The arrangement as in claim 14, wherein the disks or plates are sector-shaped and in that one or more, of the sector-shaped parts can be removed from and fixed to the element.

16. The arrangement as in claim 15, wherein the samples are tablet-shaped with a and have a diameter of about 6 mm and a thickness of about 2 mm, and in that each tablet-shaped sample can be removed from and fixed to the sector.

17. The arrangement as in claim 16, wherein the element can be rotated on a bearing and is provided with a casing for covering the samples, during transportation and storage of the cone-shaped unit.

18. The arrangement as in claim 1, wherein each bottle-shaped container in cross section has curved outer sides and inner sides, in that the inner side has a smaller radius than the outer side, and in that the inner side and a closure cap can cooperate with the inner side of a slightly curved hand, and the closure cap is accessible to the thumb and index finger of the hand, and in that the container has a width of about 35-50 mm, and the closure has a diameter of about 10-20 mm.

19. An arrangement for displaying and storing substances for construction and coloring of dental products, the arrangement comprising:
a plurality of bottles containing substances for coloring ceramic teeth; and
a cone-shaped display unit configured such that the bottles are arranged into at least two sector-shaped units, each of the at least two sector-shaped units comprising containers with substances having a different characteristic than the other sector-shaped unit, the containers in each sector-shaped unit being sub-divided into at least a first and second categories having a different characteristics from each other, and the bottles of the first category are positioned outermost in the respective sector-shaped unit with the bottles of the second category positioned to the inside of the bottles of the first category in the respective sector-shaped unit.
